(19) United States
(12) Patent Application Publication
(10) Pub. No.: US 2024/0226505 A9
(43) Pub. Date: Jul. 11, 2024

(54) Introducer Needle and Stylet for Interventional Procedures

(71) Applicant: Wisconsin Alumni Research Foundation, Madison, WI (US)

(72) Inventors: Annie M. Zlevor, Madison, WI (US); Fred T. Lee, JR., Madison, WI (US); Timothy Szczyykutowicz, Madison, WI (US); J. Louis Hinshaw, Middleton, WI (US)

(21) Appl. No.: 18/487,228

(22) Filed: Oct. 16, 2023

Related U.S. Application Data
(60) Provisional application No. 63/380,823, filed on Oct. 25, 2022.

(51) Int. Cl. A61M 25/01; A61M 25/06

(52) U.S. Cl. CPC A61M 25/0102; A61M 25/065

(57) ABSTRACT

An introducer tube/stylet needle assembly provides for an interfacial channel between the stylet and introducer tube to allowing the flow of anesthetic through the introducer tube from a syringe or the like without removal of the stylet from an introducer tube. A valve structure may automatically block gas flow through the introducer tube when the stylet is removed.

### CROSS REFERENCE TO RELATED APPLICATION

This application claims the benefit of U.S. provisional application 63/380,823 filed Oct. 25, 2022 and hereby incorporated by reference.

### STATEMENT REGARDING FEDERALLY SPONSORED RESEARCH OR DEVELOPMENT

### Background of the Invention

The present invention relates to introducer needles, for example, used for obtaining tissue biopsies and the like, and in particular to an improved introducer needle/stylet combination.

Many medical procedures utilize an introducer needle (cannula) that can be inserted through the skin of the patient to a target region for a specific intervention, for example, biopsy, injection, aspiration, drain placement, ablation, etc. Introducer needles are generally made of stainless steel (brass and titanium are two other infrequently-used metals with the former ideal for MRI-guided procedures) and are extremely simple in design with a screw-on Luer-lock often with a translucent plastic hub. During the insertion, a solid stylet is placed coaxially within the introducer needle. The stylet may also have a translucent plastic hub on the proximal end and a pointed cutting tip on the distal end which help to pierce the tissue and help prevent coring of tissue. Some stylets may also have a blunt tip that prevents coring by the introducer needle but without the cutting tip. During insertion, the plastic hub on the stylet can mate with a plastic funnel-shaped hub on the introducer needle holding the two together. Once the target is reached, the stylet can be removed in preparation for the specific intervention, for example, insertion of a biopsy device.

The process of guiding the introducer needle to the target is often facilitated by CT imaging which identifies the target location within an image slice and which then can be used to align the introducer needle with the slice using the laser of the CT machine. While this CT imaging can greatly assist in locating the stylet tip, the introduction of the foreign stylet material can create image artifacts, typically streaks, that extend outward from the stylet tip and can obscure the important regions of the image in that area.

Periodically, as the introducer needle is inserted, the stylet is removed and a local anesthetic introduced into the introducer needle to numb the tissue. When a CT scanner laser is used for alignment, removal of the stylet to allow anesthetic to be introduced can require moving the patient in and out of the CT scanner because of the limited space within the CT bore. Removal of the stylet is also a problem with other imaging guidance modalities, such as ultrasound, to the extent that it delays the intervention and can potentially alter the targeting after the needle manipulation.

When the stylet is removed for numbing or to insert the biopsy or other device, air can leak into the hollow introducer needle. This is a safety concern during lung biopsy or vascular procedures because the introduction of air can cause a potentially fatal air embolism or pneumothorax during exchange. To address this concern, physicians may place a finger over the opening of the introducer needle during the exchange process; however, this presents a risk of injury to the physician when rapidly exchanging sharpened devices such as biopsy needles and stylets.

### SUMMARY OF THE INVENTION

The present invention greatly simplifies the use of introducer needles by providing a path of anesthetic along the stylet when the stylet is in place, eliminating the need to remove the stylet and extract the patient from the CT machine, and more generally, eliminating delay and unnecessary needle manipulation during any imaging-guided procedure. In one embodiment, the introducer needle also automatically seals itself when the stylet is removed. Additional improvements include providing an improved optical target for the CT laser and reducing image artifacts during imaging of the target with the introducer needle and stylet in place through the use of a low average atomic number over the length of the stylet.

More specifically, in one embodiment, the invention provides a needle assembly having an introducer tube extending along an axis and providing a central lumen and sized for percutaneous introduction through the skin of a patient. The needle assembly includes a stylet having a sharpened tip and slidably receivable coaxially within the introducer tube to extend therethrough to expose the sharpened tip from the distal end of the introducer tube. At least one of the inner surfaces of the introducer tube and outer surface of the stylet provides an axial (longitudinal) channel allowing the flow of liquid along the channel when the stylet is inserted in the introducer tube. A flow control assembly is attached to a proximal end of the introducer tube and provides an axially extending chamber interconnecting the central lumen of the introducer tube, an introduction port adapted to receive anesthetic from a syringe or the like, and a stylet port through which the stylet may be received before entering the lumen of the introducer tube. The stylet port includes a valve blocking the passage of air through the stylet port when the stylet or other similar probe has been removed from the flow control assembly.

It is thus a feature of at least one embodiment of the invention to eliminate the need for the physician to block airflow through the introducer tube using the physician's finger during the interchange of sharp elements in the introducer tube.

The introducer tube may include at least one set of circumferential openings along its length in fluid communication with the channel when the stylet is inserted in the introducer tube.

It is thus a feature of at least one embodiment of the invention to allow the distribution of anesthetic out of several locations at the distal end of the introducer tube during its insertion.

At least one of inner surface of the introducer tube and outer surface of the stylet may provide a circumferential channel communicating among the circumferential openings when the stylet is inserted into the introducer tube.

It is thus a feature of at least one embodiment of the invention to provide improved distribution of anesthetic at different locations circumferentially around the introducer tube.

The flow control assembly may further include a guide channel providing an opening matching a cross-section of the stylet and channel for receiving the stylet and blocking the passage of fluid through the channel within the guide channel.

It is thus a feature of at least one embodiment of the invention to prevent backflow of anesthetic through the needle assembly, for example, when the stylet is being removed and the valve is not yet closed.

The stylet port and stylet hub when retained together may block fluid flow through the stylet reception port.

It is thus a feature of at least one embodiment of the invention to prevent backflow of anesthetic and back or forward flow of gases during insertion of the needle assembly when the stylet is engaged with the introducer tube.

The stylet may include a stylet hub providing a proximal surface presenting a roughened surface to provide non-specular reflection of a laser beam.

It is thus a feature of at least one embodiment of the invention to greatly improve the visibility of a CT laser during the process of aligning the needle assembly with a target assessed by a CT machine or the like.

The walls of the channel may have a greatest circumferential separation at an outer surface of the stylet.

It is thus a feature of at least one embodiment of the invention to provide a channel shape that is amenable to sealing.

The stylet may have an effective atomic number less than 20.

It is thus a feature of at least one embodiment of the invention to reduce x-ray image artifacts interfering with visualization of the target or other critical structures like blood vessels when imaging the needle assembly close to the target.

These particular objects and advantages may apply to only some embodiments falling within the claims and thus do not define the scope of the invention.

### BRIEF DESCRIPTION OF THE DRAWINGS

FIG. 1 is a perspective view of an introducer needle and stylet of the present invention with expanded portions showing the inter-fitting of the stylet within the introducer tube at a distal end during insertion, a flow control assembly providing for the introduction of anesthetic and blocking of airflow into the introducer tube, and a proximal end of the introducer tube and stylet showing an interlocking hub;

FIG. 2 is an elevational cross-section of the flow control assembly along the axis of the introducer needle showing insertion of the stylet through blocking elements to a chamber receiving anesthetic and an inset providing a cross-section through the stylet showing a channel for the passage of anesthetic along the stylet within the introducer tube;

FIG. 3 is a figure similar to that of FIG. 2 with the stylet removed and showing a closure of a valve of the blocking elements;

FIG. 4 is an elevational view of the introducer tube and stylet disassembled but aligned showing anesthetic delivery holes in the introducer tube that may be fed by the channel in the stylet; and

FIG. 5 is a perspective view of the proximal end of the stylet showing a surface for receiving a projected laser at a non-specular surface for improved visibility of the crosshairs.

### DETAILED DESCRIPTION OF THE PREFERRED EMBODIMENT

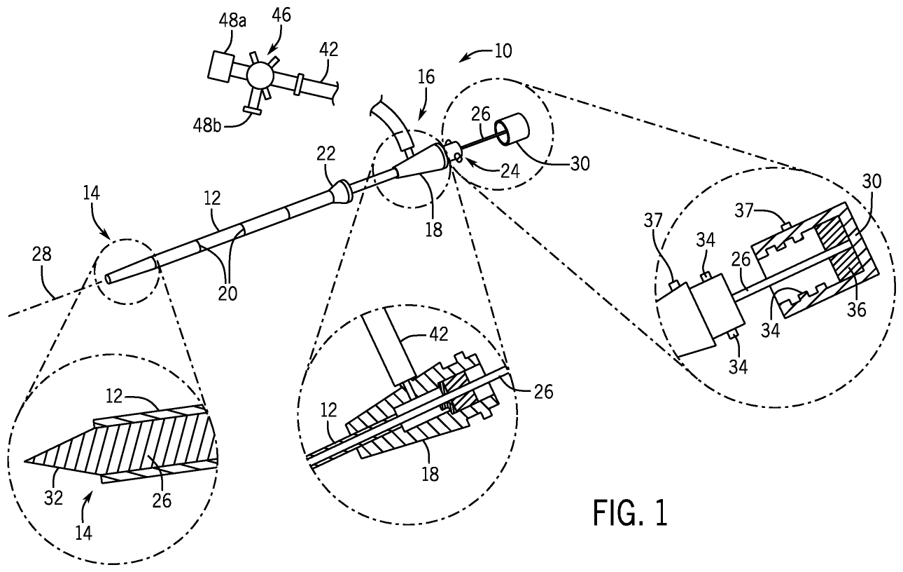

Referring now to FIG. 1, the introducer tube assembly 10 of the present invention may provide an introducer tube 12, for example, being a stainless steel tube (or a material with lower atomic number, as discussed below) typically with a gauge from 9-25 outside diameter and having a blunt distal end 14 and a proximal end 16 communicating with a flow control assembly 18. The outer surface of the introducer tube 12 may include depth markings 20 or the like and a depth slider 22 for controlling the depth of percutaneous insertion of the introducer tube 12 into the patient.

The flow control assembly 18 provides a stylet port for receiving a solid wire stylet 26 having a generally circular cross-section cut with a channel described below and extending along axis 28 aligned with the axis of the introducer tube 12. The tip of the stylet 26 at its distal end may be received through the stylet port and then may pass through the introducer tube 12. A proximal end of the stylet 26 is attached to a stylet hub 30, for example, constructed of a thermoplastic material. The stylet hub 30 may be fixed to the stylet 26 or attached pivotally so as to permit relative rotation between the stylet hub 30 and the stylet 26 but not axial separation as will be discussed in greater detail below.

When the stylet 26 is fully inserted into the introducer tube 12 a tip 32 of the stylet 26 is exposed at the distal end 14 of the introducer tube 12 to aid in piercing the tissue when sharpened, or to avoid piercing the bowel or other critical structures when a blunt tip is used. The outer diameter of the stylet 26 closely matches the inner diameter of the introducer tube 12 with a sliding fit to prevent coring of tissue or catching on tissue during the insertion process.

The stylet port and stylet hub 30 may provide for interengaging thread portions 34 allowing them to be releasably connected by relative rotational motion, for example, a quarter turn. An elastomeric gasket 36 or equivalent sealing structure may be incorporated onto one or both of the stylet port and stylet hub 30 to provide a gas tight seal when the two are connected. In one embodiment, each of the stylet port and stylet hub 30 or the stylet 26 may provide for fiducial marks 37 allowing them to be aligned with a predetermined relative rotational orientation during engagement and at other times providing an improved sealing of a channel in the stylet 26 as will be discussed below.

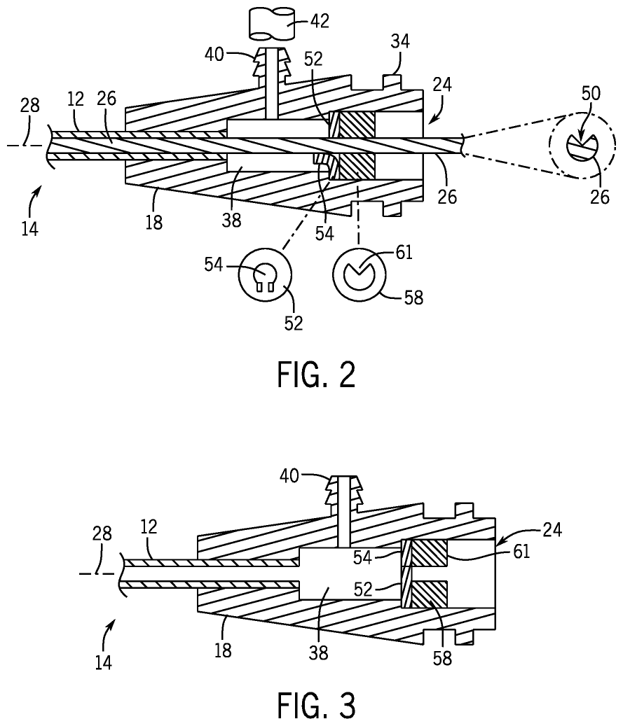

Referring also to FIG. 2, the flow control assembly 18 may provide for a central chamber 38 providing a passageway communicating between a lumen of the introducer tube 12 and a passageway of the stylet port. This chamber 38 also connects to an introduction port 40, for example, providing a Luer-lock, barbed connector or weld that may connect to standard IV tubing 42 or the like for the introduction of anesthetic or aspiration. In this regard and referring to FIG. 1, the tubing 42 may connect to a valve 46 allowing selective connection of the tubing 42 to one of two ports 48a and 48b, the former providing a standard Luer lock, for example, to allow attachment of the syringe with anesthetic or the like.

Referring now still to FIG. 2, the stylet 26 may provide for an axial channel 50 extending along its length. In one embodiment, the axial channel 50 may be a groove and having a circumferential dimension expanding monotonically as one moves radially toward the outer surface of the stylet 26. The area of the channel 50 may be, for example, at least 5% of the cross-sectional area of the inner diameter of the introducer tube 12 and provides a passage away between an inner surface of the introducer tube 12 and an outer surface of the stylet 26 along the length of the introducer tube 12, when the stylet 26 is in place, allowing for the delivery of anesthetic introduced into the chamber 38 to the patient through the introducer tube 12. It will be generally appreciated that the channel 50 may alternatively be placed in the inner surface of the introducer tube 12 to similar effect or may be contained within the style at 26 as a bore. Generally, a diameter of the chamber 38 will be much larger than the diameter of the stylet 26 allowing a free flow of liquid around stylet 26 and into the channel 50 independent of the orientation of the stylet 26.

The passageway of the stylet port may include a membrane valve 52 having a flap portion 54 elastically biased to close an opening through which the stylet 26 passes when the stylet 26 is not in place. The resilience of the flap portion 54 is sufficient to block gas flow along the axis 28 out of the stylet port and, importantly, also in the opposite direction when the stylet 26 is removed. The membrane valve 52, for example, may be constructed of a silicone rubber or the like.

Optionally, the stylet port may also include a guide channel 58 matching the cross-section of the stylet including the channel 50 to block the flow of material along the channel 50 out of the stylet port. In this regard, the guide channel 58 may include an inwardly extending tab 61 fitting closely within the channel 50 to prevent flow therethrough. The guide channel 58, for example, may be a rigid Teflon material or the like and may rotate in close proximity to the interior surface of the stylet port during rotation of the stylet hub 30 and stylet 26, or such rotation of the stylet 26 may be accommodated by pivoting of the hub 30 with respect to the stylet 26s. Alternatively, the guide channel 58 may be an elastomeric material allowing rotation of the stylet 26 by simple deformation of the tab 61. It will be appreciated that the features of the guide to channel 58 may alternatively be incorporated into the membrane valve 52.

Referring now to FIG. 3, when the stylet 26 is removed, the flap 54 blocks the chamber 38 from communication through the stylet port eliminating the need for the physician to put their finger over the opening of the stylet port during stylet removal and replacement with a biopsy device or the like. Flow through the introduction port 40 is blocked by the presence of a syringe or through the valve 46 as discussed above.

Referring now to FIG. 4, a set of circumferentially spaced openings 70 may be cut through the wall of the introducer tube 12, removed from the distal end 14, for example, by a centimeter or at least 5 mm and less than 4 cm to provide a passageway for anesthetic passing along the channel 50 to be released to tissue adjacent to the openings 70, as well as from the distal end 14 of the introducer tube 12. An optional groove 72 may be cut circumferentially in the stylet 26 to allow circumferential dispersion of material from the channel 50 to each of the openings 70. Generally, the fluid resistance through the openings 70 and through the distal end 14 will be balanced to provide a predetermined flow through each and will be much lower than the fluid resistance out of the stylet port, as implemented through the adjustment of the size and length of the channel 50 and size of the openings a 70 as will be generally understood in the art.

A distal portion 74 of the stylet 26 or the entire stylet 26 may be constructed of a low atomic number material such as aluminum, titanium, or carbon fiber typically having an effective atomic number of less than 12 in contrast to steel or the like to reduce image artifacts produced in a CT machine caused by the blockage of x-rays by high atomic number materials. Generally, the introducer tube 12 has less mass and can be a steel material; however, low atomic numbers are also contemplated for the introducer tube 12 in some embodiments, for example, by using aluminum, carbon fiber, or the like to provide an average effective atomic number of less than 20 and desirably less than 15. In some cases, all of the individual materials of the stylet may have atomic numbers less than 14.

Importantly, the inventors have recognized that the CT artifacts extending forward from the stylet are not a function simply of the material of the tip of the stylet but have contributions from material along the full length of the stylet that cause the stylet to block x-rays along a particular projection of the CT projection set aligned with the stylet axis. For this reason, in some embodiments, the entire stylet and not just the tip may be constructed of a low atomic number material (having an effective attenuation similar to a material with an atomic number less than 14). On the other hand, for the same reason, the inventors contemplate that the entire stylet and even the tip of the stylet need not be constructed of a low atomic number material if the average atomic number is sufficiently controlled over the length of the stylet to reduce attenuation of x-rays along the stylet axis, for example having an average effective atomic number of less than 20). In some cases, then, the tip of the stylet may be a high effective atomic number material improving its tracking and better allowing for sharpened surfaces followed by a low effective atomic number material in the distally extending shaft which compensates to some extent for the high atomic number tip. For example, the distal portion 74 of the stylet 26 may be a relatively hard material such as aluminum, steel, or titanium having a corresponding, relatively high effective atomic number but good mechanical properties for piercing tissue, and the remainder of the stylet 26 may be a relatively lower effective atomic number material, such as carbon fiber, to maintain an effective average atomic number of less than 20. Typically, the high atomic number tip will comprise less than 15% of the length of the stylet 26. Effective atomic number may be measured according to the teachings of Murty, R., Effective Atomic Numbers of Heterogeneous Materials. Nature 207, 398-399 (1965). https://doi.org/10.1038/207398a0.

Although the inventors do not wish to be bound by a particular theory, it is believed that the most important image artifacts, extending from the tip of the stylet 26 are produced by the cumulative blocking effect of x-rays traveling parallel to or close to parallel to the length of the stylet which is effectively opaque in that dimension and by the material closest to the tip. Accordingly, desirably the stylet has over its entire length an effective average atomic number of less than 12 and can be a metallic material over a shank offset by much lower atomic number shaft material at a tip. In some cases, the weighted average atomic number can be obtained through hollow structures.

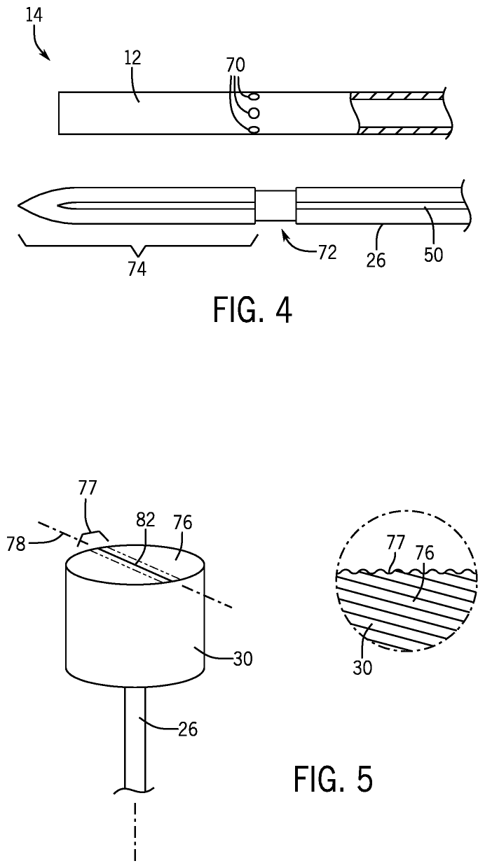

Referring now to FIG. 5, the stylet hub 30 may present a proximal surface 76 being generally perpendicular to the axis 28 which may receive a projection of a laser planes 78 forming a line of illumination at a centerline 82 on the surface 76. In this regard, the surface 76 may be given a surface roughness to produce a non-specular or diffuse reflection improving the visibility of the crosshair 82 in contrast to highly reflective surfaces ordinarily employed. Generally, for laser light of approximately 700 nm, a surface RMS roughness of 35 nm or more than 15 nm will be adequate. In addition, the thermoplastic employed for the hub 30 may be an opaque and reflective material. In one embodiment, the surface roughness may be confined to a small region 77 about the centerline 82, for example, 2 to 4 mm so as to produce a substantial brightening of the line of laser when it is close to aligned with the centerline 82 while allowing the centerline 82 to be visible at a lesser intensity anywhere along the surface 76. The centerline 82 may also be marked, for example, with a scribe or a darkened line or the like. Areas outside of this region 77 maybe smooth relative to the region 77.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom”, and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including”, and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “a microprocessor” and “a processor” or “the microprocessor” and “the processor,” can be understood to include one or more microprocessors that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

What we claim is:

1. A needle assembly comprising:
an introducer tube extending along an axis and providing a central lumen and sized for percutaneous introduction through the skin of a patient;
a stylet having a tip and being slidably receivable coaxially within the introducer tube to extend therethrough to expose the tip from a distal end of the introducer tube;
at least one of an inner surface of the introducer tube and outer surface of the stylet providing an axial channel allowing a flow of liquid along the channel when the stylet is inserted in the introducer tube; and
a flow control assembly attached to a proximal end of the introducer tube and providing an axially extending chamber interconnecting the central lumen, an introduction port adapted to receive anesthetic from a syringe or the like, and a stylet port through which the stylet may be received before entering the lumen of the introducer tube; wherein the axial channel provides a passageway between the introduction port and the distal end of the introducer tube.

2. The needle assembly of claim 1 wherein the introducer tube includes at least one set of circumferential openings along its length in fluid communication with the channel when the stylet is inserted in the introducer tube.

3. The needle assembly of claim 1 wherein at least one of the inner surface of the introducer tube and outer surface of the stylet provides a circumferential groove communicating among the circumferential openings when the stylet is inserted into the introducer tube.

4. The needle assembly of claim 1 wherein the axial channel is a groove and the flow control assembly further includes a guide channel providing an opening matching a cross-section of the stylet and channel for receiving the stylet and blocking the passage of fluid through the channel within the guide channel.

5. The needle assembly of claim 1 wherein the stylet port includes a valve blocking a passage of air through the stylet port when the stylet has been removed from the flow control assembly.

6. The needle assembly of claim 1 wherein the stylet includes a stylet hub attached to a proximal end of the stylet and wherein the stylet reception port and stylet hub provide interengaging threads for releasably retaining the stylet within the introducer tube.

7. The needle assembly of claim 6 wherein the stylet port and stylet hub when retained together block fluid flow through the stylet reception port.

8. The needle assembly of claim 1 wherein the channel is a groove in the outer surface of the stylet.

9. The needle assembly of claim 1 wherein the valve is an elastic membrane.

10. The needle assembly of claim 1 wherein the stylet includes a stylet hub providing a proximal surface presenting a roughened surface to provide non-specular reflection of a laser beam.

11. The needle assembly of claim 10 wherein the proximal surface has an RMS surface roughness of at least 35 nm.

12. The needle assembly of claim 1 further including a manually operated valve communicating between the introduction port and at least one Luer-lock port.

13. The needle assembly of claim 1 wherein the axial channel is a groove and the walls of the axial channel have a greatest circumferential separation at an outer surface of the stylet.

14. The needle assembly of claim 1 wherein the introducer needle has a gauge of 9-25 outer diameter.

15. The needle assembly of claim 1 wherein the stylet has an effective average atomic number less than 20.

16. The needle assembly of claim 15 wherein the stylet is a material selected from the group consisting of aluminum and carbon fiber.

17. The needle assembly of claim 1 wherein the channel occupies at least 5% of a cross sectional area of the introducer tube.

18. A needle assembly comprising:
an introducer tube extending along an axis and providing a central lumen and sized for percutaneous introduction through the skin of a patient;
a stylet having a tip and being slidably receivable coaxially within the introducer tube to extend therethrough to expose the tip from a distal end of the introducer tube; and
wherein the stylet has an average effective atomic number measured along the axis of less than 20.

19. The needle assembly of claim 18 wherein a distal portion of the stylet has a higher effective atomic number than a proximal portion of the stylet.

20. The needle assembly of claim 18 wherein the stylet has an average effective atomic number of greater than 6.
